(19) United States
(12) Patent Application Publication
(10) Pub. No.: US 2022/0189742 A1
(43) Pub. Date: Jun. 16, 2022

(54) DRYING UNIT AND APPARATUS FOR PROCESSING A SUBSTRATE INCLUDING A DRYING UNIT

(71) Applicant: Semes Co., Ltd., Cheonan-si (KR)

(72) Inventors: Dahyun KIM, Cheonan-si (KR); Hakjun LEE, Anan-si (KR); Yonghyun HAM, Bucheon-si (KR)

(73) Assignee: Semes Co., Ltd., Cheonan-si (KR)

(21) Appl. No.: 17/521,950

(22) Filed: Nov. 9, 2021

(30) Foreign Application Priority Data
Dec. 16, 2020 (KR) 10-2020-0176535

(51) Int. Cl. H01J 37/32; H05B 3/00; H05B 1/02; F27B 17/00; F26B 3/28

(52) U.S. Cl. CPC H01J 37/32522; H05B 3/0047; H05B 2203/032; F27B 17/0025; F26B 3/28; H05B 1/0233

(57) ABSTRACT

An apparatus for processing a substrate may include a processing module including at least one process chamber for processing a desired process on a substrate, an index module for transferring the substrate from an outside into the processing module, and a drying unit for removing a moisture or undesired gases from the at least one process chamber. The drying unit may remove the moisture or the undesired gases from components newly installed in the at least one process chamber.

### CROSS-REFERENCE TO RELATED APPLICATION

This application claims priority to Korean Patent Application No. 10-2020-0176535 filed on Dec. 16, 2020 in the Korean Intellectual Property Office (KIPO), the contents of which are herein incorporated by reference in its entirety.

### BACKGROUND

### 1. Field

Example embodiments of the invention relate to a drying unit and an apparatus for processing a substrate including the drying unit. More particularly, example embodiments of the invention relate to a drying unit for effectively removing moisture or undesired gases from a process chamber and an apparatus for processing a substrate including such a drying unit.

### 2. Related Technology

In general, an integrated circuit device or a display device may be manufactured using an apparatus for processing a substrate, which may include various process chambers such as a deposition chamber, a sputtering chamber, an etching chamber, a cleaning chamber, a drying chamber, etc. To manufacture the integrated circuit device or the display device, desired processes may be performed on a substrate placed in the process chamber while maintaining an inside of the process chamber in a vacuum state.

Components installed in the process chamber are replaced periodically or as occasion demands since the components may be damaged or worn in various processes for manufacturing the integrated circuit device or the display device. The components newly installed in the process chamber can be introduced into the process chamber while the components are exposed to an external environment including the atmosphere. However, moisture or undesired gases may be discharged from the components newly installed in the process chamber while the various processes are performed on a substrate loaded in the process chamber. Such moisture or undesired gases may cause failures of the processes performed in the process chamber such that the reliability of the integrated circuit device or the display device may be reduced. Considering these problems, a separate drying device can be used to remove the moisture or the undesired gases from components newly installed in the process chamber, however, the throughput of the integrated circuit device or the display device may be lowered because of relatively processing time and also the cost for manufacturing the integrated circuit device or the display device may be increased.

### SUMMARY

One aspect of the invention provides a drying unit capable of rapidly and effectively removing moisture or undesired gases from components newly installed in a process chamber.

Another aspect of the invention provides a process chamber including a drying unit capable of rapidly and effectively removing moisture or undesired gases from components newly installed in the process chamber.

Still another aspect of the invention provides an apparatus for processing a substrate including a drying unit capable of rapidly and effectively removing moisture or undesired gases a process chamber.

In one aspect of the invention, there is provided a drying unit for removing a moisture or undesired gases from a process chamber. The drying unit may include at least one light source for emitting a light; and a control member for providing a power to the at least one light source and controlling the at least one light source.

In example embodiments, the process chamber may include an etching chamber, a deposition chamber, a sputtering chamber, a coating chamber, a developing chamber, a cleaning chamber or a drying chamber. For example, the drying unit may remove the moisture or the undesired gases from components newly installed in the process chamber.

In example embodiments, the drying unit may have the substantially same shape and the substantially same dimension as a shape and a dimension of a substrate loaded in the process chamber. In this case, the drying unit may be loaded into the process chamber and may be unloaded from the process chamber using a transferring member that loads the substrate into the process chamber and unloads the substrate from the process chamber.

In example embodiments, the at least one light source may include a transparent container and a heavy hydrogen gas filled in the transparent container. For example, the transparent container may include a transparent ceramic selected from the group consisting of quartz, transparent alumina and transparent lead lanthanum zirconate titanate (PLZT). The at least one light source may emit an ultraviolet (UV) light having a wavelength of about 100 nm to about 400 nm.

In example embodiments, the control member may be attached to a bottom of the at least one light source, and may include a battery element for providing a power to the at least one light source and a control circuit for controlling the at least one light source.

In some example embodiments, the at least one light source may include a first light source and a second light source and the control member is disposed between the first light source and the second light source.

In some example embodiments, the control member may include a battery element for providing a power to each of the first and second light sources and a control circuit for controlling each of the first and second light sources.

In some example embodiments, the first light source and the second light source may emit UV lights having the substantially same wavelength range.

In other example embodiments, the first light source and the second light source may emit UV lights having different wavelength ranges, respectively.

In another aspect of the invention, there is provided a process chamber for processing a desired process on a substrate. The process chamber may include a housing providing a processing space therein, a supporting unit installed in the housing for supporting the substrate, and a drying unit placed on the supporting unit instead of the substrate wherein the drying unit may remove a moisture or undesired gases from components newly installed in the process chamber.

In example embodiments, the drying unit may include at least one light source for emitting a light and a control member for providing a power to the at least one light source and controlling the at least one light source.

In example embodiments, the at least one light source may include a transparent container and a heavy hydrogen gas filled in the transparent container.

In some example embodiments, the at least one light source may include a first light source and a second light source and the control member is disposed between the first light source and the second light source.

In some example embodiments, the first light source and the second light source may emit UV lights having a same wavelength range, or UV lights having different wavelength ranges, respectively.

In still another aspect of the invention, there is provided an apparatus for processing a substrate. The apparatus for processing a substrate may include a processing module including at least one process chamber for processing a desired process on a substrate, an index module for transferring the substrate from an outside into the processing module, and a drying unit for removing a moisture or undesired gases from the at least one process chamber.

In example embodiments, the drying unit may include at least one light source for emitting a light and a control member for providing a power to the at least one light source and controlling the at least one light source.

According to example embodiments of the invention, the drying unit may rapidly and effectively remove the moisture or the undesired gases from various components newly installed in the process chamber without any separate drying device. Therefore, failures of various processes performed in the process chamber may be prevented and also reliability of an integrated circuit device or a display device may be improved. Further, the apparatus for processing a substrate including the drying unit may enhance the throughput of the integrated circuit device or the display device.

### BRIEF DESCRIPTION OF THE DRAWINGS

Example embodiments will be more clearly understood from the following detailed description taken in conjunction with the accompanying drawing. The following figures represent non-limiting, example embodiments as described herein.

FIG. 1 is a plan view illustrating an apparatus for processing a substrate in accordance with example embodiments of the invention.

FIG. 2 is a cross-sectional view illustrating a process chamber of an apparatus for processing a substrate in accordance with example embodiments of the invention.

FIG. 3 is a perspective view illustrating a drying unit of an apparatus for processing a substrate in accordance with example embodiments of the invention.

FIG. 4 is a schematic cross-sectional view illustrating an operation of a drying unit loaded in a process chamber in accordance with example embodiments of the invention.

FIG. 5 is a perspective view illustrating a drying unit of an apparatus for processing a substrate in accordance with some example embodiments of the invention.

### DESCRIPTION OF EMBODIMENTS

Various embodiments will be described more fully hereinafter with reference to the accompanying drawings, in which some embodiments are shown. The invention may, however, be embodied in many different forms and should not be construed as limited to the embodiments set forth herein. Rather, these embodiments are provided so that this description will be thorough and complete, and will fully convey the scope of the invention to those skilled in the art. In the drawings, the sizes and relative sizes of layers and regions may be exaggerated for clarity.

It will be understood that when an element or layer is referred to as being “on,” “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numerals refer to like elements throughout. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the invention.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (for example, rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a,” “an” and “the” are intended to include a plurality of forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments are described herein with reference to cross-sectional illustrations that are schematic illustrations of idealized embodiments (and intermediate structures). As such, variations from the shapes of the illustrations as a result, for example, of manufacturing techniques and/or tolerances, are to be expected. Thus, embodiments should not be construed as limited to the particular shapes of regions illustrated herein but are to include deviations in shapes that result, for example, from manufacturing. For example, an implanted region illustrated as a rectangle will, typically, have rounded or curved features and/or a gradient of implant concentration at its edges rather than a binary change from implanted to non-implanted region. Likewise, a buried region formed by implantation may result in some implantation in the region between the buried region and the face through which the implantation takes place. Thus, the regions illustrated in the figures are schematic in nature and their shapes are not intended to illustrate the actual shape of a region of a device and are not intended to limit the scope of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, example embodiments of the invention will be described in detail with reference to the accompanying drawings. Like elements or components can be indicated by like reference numerals throughout the drawings, and the repeated explanations of like elements or components may be omitted.

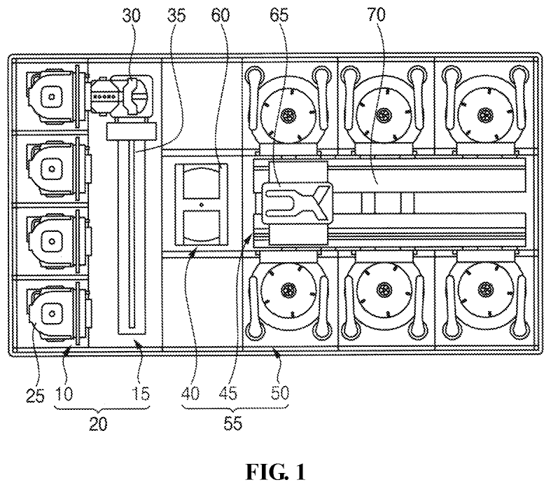

FIG. 1 is a plan view illustrating an apparatus for processing a substrate in accordance with example embodiments of the invention.

Referring to FIG. 1, an apparatus for processing a substrate may include an index module 20 and a processing module 55.

The index module 20 may transfer a substrate into the processing module 55 from an outside and the processing module 55 may perform a predetermined process on the substrate. Here, the substrate may be used to manufacture an integrated circuit device or a display device. For example, the substrate may include a silicon wafer, a glass substrate, an organic substrate, etc.

In example embodiments, the index module 20 may include a load chamber 10 and a transferring frame 15. A carrier 25 for receiving the substrate may be loaded in the load chamber 10. For example, a front opening unified pod (FOUP) may be used as the carrier 25. The carrier 25 may be transferred into the load chamber 10 from the outside or from the load chamber 10 to the outside using an overhead transfer (OHT).

The transferring frame 15 may transfer the substrate between the carried 25 loaded in the process chamber 10 and the processing module 55. The transferring frame 15 may include an index robot 30 and an index rail 35.

The index robot 30 may move along the index rail 35 and may transfer the substrate between the index module 20 and the processing module 55. For example, the index robot 30 may transfer the substrate between the carrier 25 and a buffer slot 60 while moving on the index rail 35.

As illustrated in FIG. 1, the processing module 55 may perform the predetermined process including, but not limited to, a deposition process, an etching process, a sputtering process, a cleaning process or a drying process, on the substrate. In this case, the processing module 55 may include a buffer chamber 40, a transfer chamber 45, a process chamber 50, a control unit (not illustrated), etc.

The substrate transferred between the index module 20 and the processing module 55 may wait in the buffer chamber 40 for the predetermined process. A buffer slot 60 receiving the substrate thereon may be disposed in the buffer chamber 40. For example, a plurality of buffer slots 60 may be disposed in the buffer chamber 40 such that a plurality of substrates may be placed in the buffer chamber 40.

The transfer chamber 45 may transfer the substrate between the buffer chamber 40 and the process chamber 50. The transfer chamber 45 may include a transferring robot 65 and a transferring rail 70. The transferring robot 65 may move along the transferring rail 70 such that the transferring robot 65 may transfer the substrate between the buffer chamber 40 and the process chamber 50. For example, as the transferring robot 65 may move on the transferring rail 70, the transferring robot 65 may transfer the substrate located on the buffer slot 60 into the process chamber 50.

In example embodiments, the apparatus for processing a substrate may include a plurality of process chambers 50. For example, the plurality of process chambers 50 may include an etching chamber, a deposition chamber, a sputtering chamber, a coating chamber, a developing chamber, a cleaning chamber or a drying chamber for performing various processes for manufacturing an integrated circuit device including a semiconductor device or a display device including a flat panel display device.

A desired process including, but not limited to, a deposition process, a sputtering process, an etching process, a developing process, a cleaning process, or a drying process, may be executed in the process chambers 50. In this case, each of the process chambers 50 may have a door opened and closed for loading and unloading the substrate into or from each process chamber 50.

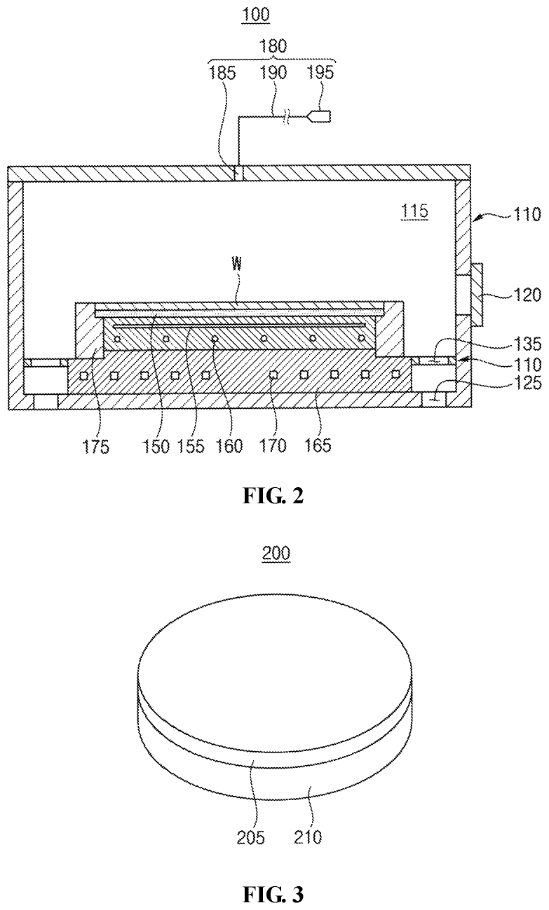

FIG. 2 is a cross-sectional view illustrating a process chamber of an apparatus for processing a substrate in accordance with example embodiments of the invention. In FIG. 2, the process chamber of the apparatus for processing a substrate may be a plasma etching chamber.

Referring to FIG. 2, the process chamber 100 may include a housing 110, a supporting unit, a gas supply unit 180, a plasma supply unit (not illustrated), a discharging unit, etc.

The housing 110 may provide a processing space 115 in which a desired process such as an etching process may be performed on a substrate W. The housing 110 may have a substantially cylindrical shape. For example, the housing 110 may be composed of metal such as aluminum or aluminum alloy. An opening may be provided at one side of the housing 110 for loading and unloading the substrate W into and from the housing 110. Such opening may be opened and closed by a door 120. A hole 125 may be provided at a bottom of the housing 110. A vacuum member (not illustrated) may be connected to the housing 110 through the hole 125 such that the processing space 115 may be maintained in a substantial vacuum state by the vacuum member, for example, a vacuum pump.

The supporting unit may be disposed in the processing space 110 and may support the substrate W in the etching process. For example, the supporting unit may include an electrostatic chuck capable of supporting the substrate W utilizing an electrostatic force. Alternatively, the supporting unit may support the substrate W by clamping the substrate W.

When the supporting unit includes the electrostatic chuck, the supporting unit may include a dielectric plate 150, an inner electrode 155, a heater 160, a base 165, a cooling flow path 170, a focus ring 175, etc.

The dielectric plate 150 may be composed of a dielectric material and the substrate W may be placed on the dielectric plate 150. The dielectric plate 150 may have a substantially circular plate shape. The dielectric plate 150 may have a diameter or a width substantially the same as a diameter or a width of the substrate W, or may have a diameter or a width substantially smaller than a diameter or a width of the substrate W. However, the dimensions of such dielectric plate 150 may vary in accordance with the dimensions of the substrate W to be processed.

The inner electrode 155 may be buried in the dielectric plate 150 and may be coupled to a power source (not illustrated). Thus, a predetermined voltage may be applied to the inner electrode 155 from the power source so that the electrostatic force may be generated between the dielectric plate 150 and the substrate W. The heater 160 may be installed in the dielectric plate 150 for heating the substrate W. The heater 160 may be disposed under the inner electrode 155. For example, the heater 160 may include a spiral coil.

As illustrated in FIG. 2, the base 165 may be coupled to the dielectric plate 150 such that the base 165 may stably support the dielectric plate 150. In example embodiments, the base 165 may have a step structure in which a central portion is substantially higher than a peripheral portion. The central portion of the base 165 may have a size substantially the same as that of the dielectric plate 150. For example, the base 165 may be composed of metal.

The cooling flow path 170 may be provided in the base 165. The cooling flow path 170 may provide a passage in which a cooling fluid may be circulated. For example, the cooling flow path 170 may have a spiral shape.

The base 165 may be electrically connected to an external high frequency power source (not illustrated). The high frequency power source may apply a predetermined power to the base 165 such that a plasma generated from a process gas in the housing 110 by the applied power may be directed toward the base 165.

A least one focus ring 175 may be disposed on the peripheral portion of the base 165. The focus ring 175 may concentrate the plasma generated from the process gas on the substrate W. The focus ring 175 may substantially surround the dielectric plate 150 and the substrate W. The focus ring 175 may have a step structure in which a peripheral portion may upwardly protrude than a central portion. Here, the dielectric plate 150 and the substrate W may be positioned over the central portion of the focus ring 175. The substrate W may be located in place by the focus ring 175 having the above structure. The focus ring 175 may extend a region where an electric field is generated in the processing space 115 such that the substrate W may be located at a center of the processing space 115 in which the plasma is formed.

Referring now to FIG. 2, the gas supply unit 180 may provide the process gas onto the substrate W supported by the supporting unit. The gas supply unit 180 may include a gas reservoir 195 and a supply line 190. The supply line 190 may connect the gas reservoir 195 to a gas inlet port 185 provided at an upper portion of the housing 110. The process gas stored in the gas reservoir 195 may pass through the supply line 190 and the gas inlet port and then may enter the processing space 115 of the housing 110.

The plasma supply unit may convert the process gas introduced in the housing 110 from the gas supply unit 180 into the plasma. For example, the plasma supply unit may include a plasma source such as an induced-coupled plasma (ICP) source. The plasma source of the plasma supply unit may include an antenna and an external power source. Here, the external power source may apply a power to the antenna. Thus, a discharge region may be formed in the processing space 115 and the process gas introduced in the discharge region may be converted into the plasma.

The discharging unit may be disposed in the processing space 115 and may be adjacent to the supporting unit. The discharging unit may include a plurality of discharging holes 135 and the plasma may be partially discharged through the discharging holes 135 such that the plasma may be uniformly distributed in the entire processing space 115. For example, the discharging unit may have a substantial ring shape.

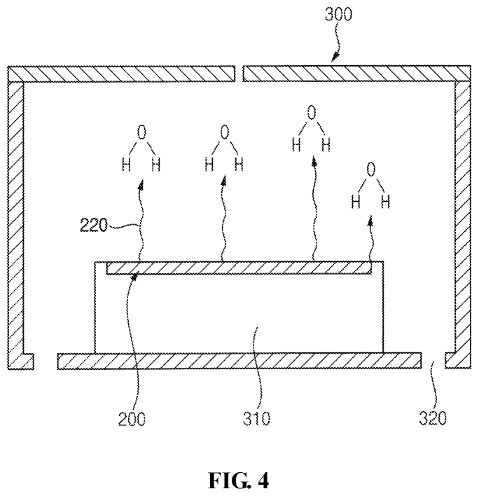

FIG. 3 is a perspective view illustrating a drying unit of an apparatus for processing a substrate in accordance with example embodiments of the invention. FIG. 4 is a schematic cross-sectional view illustrating an operation of a drying unit loaded in a process chamber in accordance with example embodiments of the invention.

Referring to FIG. 3, the drying unit 200 may include a light source 205 and a control member 210 which may be attached to a bottom of the light source 205. The control member 210 may include a battery element for providing a power to the light source 205 and a control circuit for controlling the battery element and the light source 205.

As illustrated in FIG. 4, the drying unit 200 may have a shape and a dimension substantially the same as a shape and a dimension of the substrate W on which the desired process such as the plasma etching process may be performed in the process chamber 300 having the above described configuration. For example, when the substrate W is a wafer having a diameter of about 300 mm, the drying unit 200 may have a circular plate shape having a diameter of about 300 mm. However, the drying unit 200 may have a shape and a dimension varied in accordance with the shape and the dimension of the substrate W placed on a supporting unit 310 disposed in the process chamber 300.

When the drying unit 200 has the shape and the dimension substantially the same as the shape and the dimension of the substrate W, the drying unit 200 may replace the substrate W located on the supporting unit 310. In other words, the drying unit 200 may be loaded into the process chamber 300 and unloaded from the process chamber 300 instead of the substrate W before the substrate W is loaded into the process chamber 300 or after the substrate W is unloaded from the process chamber 300. For example, the drying unit 200 may be loaded into the process chamber 300 and may be unloaded from the process chamber 300 by a transferring member such as the robot arm for the loading/unloading of the substrate W. Accordingly, in example embodiments, a separate device may not be required to load the drying unit 200 into the process chamber 300 and to unload the drying unit 200 from the process chamber 300.

According to example embodiments, the light source 205 may include a transparent container and a heavy hydrogen gas filled in the transparent container. For example, the transparent container may include a transparent ceramic including, but not limited to, quartz, transparent alumina, or transparent lead lanthanum zirconate titanate (PLZT). The light source 205 may emit an ultraviolet (UV) light having a wavelength range of about 100 nm to about 400 nm, preferably about 115 nm to about 399 nm.

In the process chamber 100, the components such as the above-described housing 110, the supporting unit 310, the gas supply unit 180, the plasma supply unit and/or the discharging unit may be replaced periodically or as occasion demands. In this case, new components including a housing, a supporting unit, a gas supply unit, a plasma supply unit and/or a discharging unit may be installed in the process chamber 300, and then the drying unit 200 may be loaded in the process chamber 300 before the substrate W is placed in the process chamber 300. After an inside of the process chamber 300 may be maintained in a vacuum state using a vacuum member (not illustrated) such as a vacuum pump connected to the hole 320 of the process chamber 300, the UV light having the above wavelength range may be emitted from the light source 205 by the control member 210 of the drying unit 200. The UV light generated from the light source 205 may effective remove moisture or other undesired gases from the new components of the process chamber 300. Therefore, the performance of the process chamber 300 may be enhanced and the lifetime of the process chamber 300 may be increased.

In general, water molecule (H₂O) may be decomposed by a light such as the UV light having the wavelength range of about 110 nm to about 150 nm. Therefore, without any additional drying device, the moisture or the gases absorbed on the new components installed in the process chamber 300 may be rapidly and effectively removed using the drying unit 200 only. The removed moisture or the gases may be discharged from the process chamber 300 through the hole 320 of the process chamber 300.

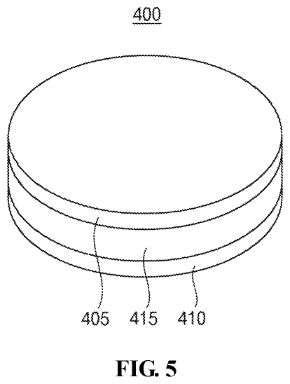

FIG. 5 is a perspective view illustrating a drying unit of an apparatus for processing a substrate in accordance with some example embodiments of the invention.

As illustrated in FIG. 5, a drying unit 400 may include a first light source 405, a second light source 410 and a control member 415. The control member 415 may be disposed between the first light source 405 and the second light source 410. The control member 415 may include a battery element for providing power to the first and second light sources 405 and 410, and a control circuit for controlling the first light source 405, the second light source 410 and the battery element. Such drying unit 400 may have a shape and a dimension substantially the same as the shape and the dimension of the substrate W.

In some example embodiments, each of the first light source 405 and the second light source 410 may include a transparent container and a heavy hydrogen gas filled in the transparent container. UV lights having the substantially same wavelength range may be emitted from the first light source 405 and the second light source 410, respectively. Therefore, the moisture or the adsorbed gas may be more rapidly and effectively from the new components installed in the process chamber 300. Alternatively, the first light source 405 and the second light source 410 may include transparent containers filled with different gases, respectively. Thus, UV lights having different wavelength ranges may be emitted from the first light source 405 and the second light source 410, respectively. In this case, the moisture or the undesired gases may be more rapidly and effectively removed from components newly installed in the process chamber 300 in accordance with the types of the new components.

According to example embodiments of the invention, the drying unit may rapidly and effectively remove the moisture or other gases from the various components newly installed in the process chamber without any separate drying device. Therefore, failures of various processes performed in the process chamber may be prevented and also the reliability of the integrated circuit device or the display device may be improved. Further, the apparatus for processing a substrate including the drying unit may enhance the throughput of the integrated circuit device or the display device.

The foregoing is illustrative of embodiments and is not to be construed as limiting thereof. Although a few embodiments have been described, those skilled in the art will readily appreciate that many modifications are possible in the embodiments without materially departing from the novel teachings and advantages of the invention. Accordingly, all such modifications are intended to be included within the scope of the invention as defined in the claims. In the claims, means-plus-function clauses are intended to cover the structures described herein as performing the recited function and not only structural equivalents but also equivalent structures. Therefore, it is to be understood that the foregoing is illustrative of various embodiments and is not to be construed as limited to the specific embodiments disclosed, and that modifications to the disclosed embodiments, as well as other embodiments, are intended to be included within the scope of the appended claims.

What is claimed is:

1. A drying unit for removing a moisture or undesired gases from a process chamber, which comprises:
at least one light source for emitting a light; and
a control member for providing a power to the at least one light source and controlling the at least one light source.

2. The drying unit of claim 1, wherein the process chamber includes an etching chamber, a deposition chamber, a sputtering chamber, a coating chamber, a developing chamber, a cleaning chamber or a drying chamber.

3. The drying unit of claim 2, wherein the drying unit removes the moisture or the undesired gases from components newly installed in the process chamber.

4. The drying unit of claim 2, wherein the drying unit has a same shape and a same dimension as a shape and a dimension of a substrate loaded in the process chamber.

5. The drying unit of claim 4, wherein the drying unit is loaded into the process chamber and unloaded from the process chamber using a transferring member that loads the substrate into the process chamber and unloads the substrate from the process chamber.

6. The drying unit of claim 1, wherein the at least one light source includes a transparent container and a heavy hydrogen gas filled in the transparent container.

7. The drying unit of claim 6, wherein the transparent container includes a transparent ceramic selected from the group consisting of quartz, transparent alumina and transparent lead lanthanum zirconate titanate (PLZT).

8. The drying unit of claim 6, wherein the at least one light source emits an ultraviolet (UV) light having a wavelength of about 100 nm to about 400 nm.

9. The drying unit of claim 1, wherein the control member is attached to a bottom of the at least one light source and includes a battery element for providing a power to the at least one light source and a control circuit for controlling the at least one light source.

10. The drying unit of claim 1, wherein the at least one light source includes a first light source and a second light source and the control member is disposed between the first light source and the second light source.

11. The drying unit of claim 10, wherein the control member includes a battery element for providing a power to each of the first and second light sources and a control circuit for controlling each of the first and second light sources.

12. The drying unit of claim 10, wherein the first light source and the second light source emit UV lights having a same wavelength range.

13. The drying unit of claim 10, wherein the first light source and the second light source emit UV lights having different wavelength ranges, respectively.

14. A process chamber for processing a desired process on a substrate, which comprises:
a housing providing a processing space therein;
a supporting unit installed in the housing for supporting the substrate; and
a drying unit placed on the supporting unit instead of the substrate, the drying unit removing a moisture or undesired gases from components newly installed in the process chamber.

15. The process chamber of claim 14, wherein the drying unit includes at least one light source for emitting a light and a control member for providing a power to the at least one light source and controlling the at least one light source.

16. The process chamber of claim 15, wherein the at least one light source includes a transparent container and a heavy hydrogen gas filled in the transparent container.

17. The process chamber of claim 16, wherein the at least one light source includes a first light source and a second light source and the control member is disposed between the first light source and the second light source.

18. The process chamber of claim 17, wherein the first light source and the second light source emit UV lights having a same wavelength range, or UV lights having different wavelength ranges, respectively.

19. An apparatus for processing a substrate, which comprises:
a processing module including at least one process chamber for processing a desired process on a substrate;
an index module for transferring the substrate from an outside into the processing module; and
a drying unit for removing a moisture or undesired gases from the at least one process chamber.

20. The apparatus for processing a substrate of claim 19, wherein the drying unit includes at least one light source for emitting a light and a control member for providing a power to the at least one light source and controlling the at least one light source.
